(19) United States
(12) Patent Application Publication
(10) Pub. No.: US 2014/0066732 A1
(43) Pub. Date: Mar. 6, 2014

(54) SYSTEM AND METHOD FOR DETERMINING CARDIAC OUTPUT

(71) Applicants: Paul Stanley Addison, Edinburgh (GB); James Nicholas Watson, Dunfermline (GB)

(72) Inventors: Paul Stanley Addison, Edinburgh (GB); James Nicholas Watson, Dunfermline (GB)

(73) Assignee: Nellcor Puritan Bennett LLC, Boulder, CO (US)

(21) Appl. No.: 13/597,501

(22) Filed: Aug. 29, 2012

(51) Int. Cl. A61B 5/0205; A61B 5/021; A61B 6/00

(52) U.S. Cl. 600/324; 600/479

(57) ABSTRACT

A system is configured to determine cardiac output of a patient. The system may include a first sub-system configured to detect a first physiological signal, and a second sub-system configured to detect a second physiological signal that differs from the first physiological signal. The first and second sub-systems may be separate and distinct from one another. The system may also include a cardiac output determination module that is configured to determine the cardiac output based, at least in part, on the first and second physiological signals.

### FIELD

Embodiments of the present disclosure generally relate to physiological signal processing and, more particularly, to processing physiological signals to determine the cardiac output of a patient.

### BACKGROUND

Blood pressure represents a measurement that quantifies a pressure exerted by circulating blood upon walls of blood vessels. In general, blood pressure is an example of a principal vital sign. Typically, blood pressure may be measured through use of a sphygmomanometer, or blood pressure cuff, and a stethoscope. However, blood pressure may also be detected through an arterial line, for example.

Blood pressure signals may be used to determine a cardiac output of a patient. In general, a parameter derived solely from the blood pressure waveform may be used with respect to a predictive model in order to yield information related to cardiac output. However, the predictive model may not account for variable physiological parameters. As such, determining cardiac output by analyzing a blood pressure signal or waveform may lead to erroneous predictions regarding cardiac output, which may, in turn, lead to false diagnoses, for example.

### SUMMARY

Embodiments of the present disclosure provide systems and methods of accurately and reliably determining cardiac output. Embodiments of the present disclosure provide systems and methods of using multiple sub-systems, such as a blood pressure sub-system and a photoplethsymogram (PPG) sub-system, to independently derive physiological data, parameters, and/or the like from separate and distinct physiological signals, for example. Instead of using models that attempt to predict cardiac output, embodiments of the present disclosure may yield actual, reliable, and accurate measurements of cardiac output.

Certain embodiments provide a system for determining cardiac output of a patient. The system may include a first sub-system configured to detect a first physiological signal, a second sub-system configured to detect a second physiological signal that differs from the first physiological signal, and a cardiac output determination module. The first and second sub-systems are separate and distinct from one another. The cardiac output determination module is configured to determine the cardiac output based, at least in part, on the first and second physiological signals.

In an embodiment, the first sub-system includes a blood pressure sub-system and the first physiological signal comprises a blood pressure signal, and the second sub-system includes a photoplethsymogram (PPG) sub-system and the second physiological signal includes a PPG signal. The cardiac output determination module may be configured to determine driving pressure from the blood pressure signal. The cardiac output determination module may be configured to determine resistance to flow through an analysis of the PPG signal and the blood pressure signal.

The blood pressure sub-system may include a blood pressure monitor operatively connected to a blood pressure detection device. The blood pressure detection device may be one or more of a non-invasive, minimally-invasive, or invasive blood pressure detection device. The PPG sub-system may include a pulse oximetry sub-system.

The cardiac output determination module may be configured to determine the cardiac output based on an analysis of at least a first parameter of the first physiological signal and at least a second parameter of the second physiological signal. The first parameter(s) may include one or more of a mean arterial pressure, a systolic pressure, or a diastolic pressure. The second parameter(s) may include one or more of a change in amplitude, baseline, or frequency of the first and second physiological signals.

The cardiac output determination module may be configured to determine cardiac output based on a driving pressure determined from the first physiological signal, and a peripheral resistance determined from the first and second physiological signals. The cardiac output determination module may be configured to determine a change in cardiac output over time based on a change in the first physiological signal over time and a relative change in the first and second physiological signals over time. The cardiac output determination module may be configured to form a ratio of a change in the first physiological signal over time with respect to a change in the second physiological signal over time.

Certain embodiments of the present disclosure provide a method of determining cardiac output of a patient. The method may include detecting a first physiological signal of the patient with a first sub-system, detecting a second physiological signal of the patient with a second sub-system that is separate and distinct from the first sub-system, wherein the second physiological signal differs from the first physiological signal, and determining the cardiac output based, at least in part, on the first and second physiological signals, with a cardiac output determination module.

Certain embodiments of the present disclosure provide a tangible and non-transitory computer readable medium that includes one or more sets of instructions configured to direct a computer to: detect a first physiological signal of a patient with a first sub-system, detect a second physiological signal of the patient with a second sub-system that is separate and distinct from the first sub-system, wherein the second physiological signal differs from the first physiological signal, and determine the cardiac output based, at least in part, on the first and second physiological signals, with a cardiac output determination module.

Certain embodiments of the present disclosure provide a system and method of determining cardiac output and/or changes in cardiac output that may account for peripheral resistance, in contrast to previous model-based systems and methods. Embodiments of the present disclosure may be more accurate and reliable than previous systems that estimated cardiac output based solely on blood pressure, for example. Embodiments of the present disclosure provide a system and method of determining cardiac output that may be used as an accuracy check with respect to other systems and methods of determining cardiac output.

Certain embodiments may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

FIG. 1 illustrates a simplified block diagram of a system for determining cardiac output, according to an embodiment.

FIG. 2 illustrates an isometric view of a photoplethysmogram (PPG) system, according to an embodiment.

FIG. 3 illustrates a simplified block diagram of a PPG system, according to an embodiment.

FIG. 4 illustrates a PPG signal over time, according to an embodiment.

FIG. 5 illustrates a simplified block diagram of a blood pressure sub-system, according to an embodiment.

FIG. 6 illustrates a simplified front view of a blood pressure sub-system, according to an embodiment.

FIG. 7 illustrates a simplified lateral view of a blood pressure detection device, according to an embodiment.

FIG. 8 illustrates a blood pressure signal over time, according to an embodiment.

FIG. 9a illustrates a simplified view of a blood vessel with a PPG detector and a blood pressure detection device detecting PPG signals and blood pressure signals, respectively, at a first time, according to an embodiment of the present disclosure.

FIG. 9b illustrates a simplified view of a blood vessel with a PPG detector and a blood pressure detection device detecting PPG signals and blood pressure signals, respectively, at a second time, according to an embodiment of the present disclosure.

FIG. 10 illustrates a flow chart of a method of determining cardiac output, according to an embodiment.

FIG. 11 illustrates a flow chart of a method of determining changes in cardiac output over time, according to an embodiment.

### DETAILED DESCRIPTION

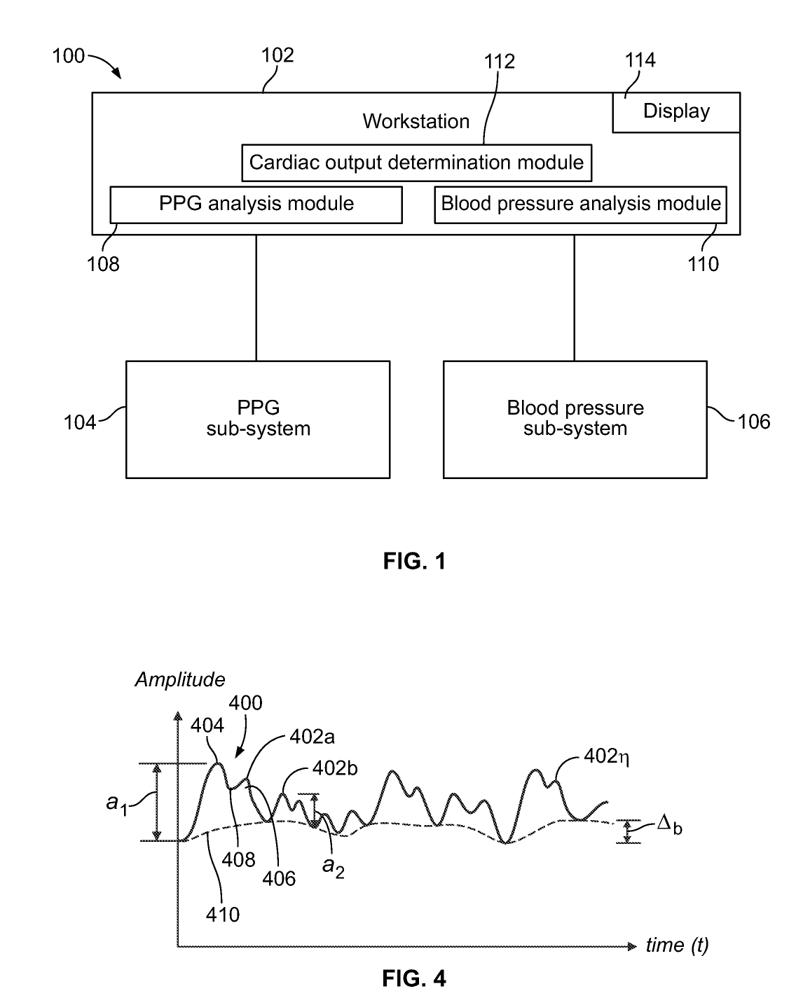

FIG. 1 illustrates a simplified block diagram of a system 100 for determining cardiac output, according to an embodiment. The system 100 may include a workstation 102 operatively connected to a photoplethsymogram (PPG) sub-system 104 and a blood pressure sub-system 106. The workstation 102 may be operatively connected to each of the PPG sub-system 104 and the blood pressure sub-system 106 through cables, wireless connections, and/or the like.

The workstation 102 may be or otherwise include one or more computing devices, such as standard computer hardware. The workstation 102 may include one or more modules and control units, such as processing devices that may include one or more microprocessors, microcontrollers, integrated circuits, memory, such as read-only and/or random access memory, and the like. For example, the workstation 102 may include a PPG analysis module 108, a blood pressure analysis module 110, and a cardiac output determination module 112. The PPG analysis module 108 may be configured to analyze a PPG signal or waveform received from the PPG sub-system 104. The blood pressure analysis module 110 may be configured to analyze a blood pressure signal or waveform received from the blood pressure sub-system 106. The cardiac output determination module 112 may be configured to determine cardiac output based on signals analyzed by the PPG analysis module 18 and the blood pressure module 110.

While shown as separate and distinct modules, the PPG analysis module 108, the blood pressure analysis module 110, and the cardiac output determination module 112 may alternatively be integrated into a single module, processor, controller, integrated circuit or the like. For example, the cardiac output determination module 112 may include the PPG analysis module 108 and the blood pressure analysis module 110. Additionally, the PPG analysis module 108 may be part of the PPG sub-system 104, while the blood pressure analysis module 110 may be part of the blood pressure sub-system 106, instead of being separately and distinctly part of the workstation 102. In such an embodiment, fully-analyzed PPG and blood pressure signals may be sent to the cardiac output determination module 112 from the PPG sub-system 104 and the blood pressure sub-system 106, respectively.

The workstation 102 may also include a display 114, such as a cathode ray tube display, a flat panel display, such as a liquid crystal display (LCD), light-emitting diode (LED) display, a plasma display, or any other type of monitor. The workstation 102 may be configured to calculate physiological parameters and to show information from the PPG sub-system 104, the blood pressure sub-system 106, and/or from other medical monitoring devices or systems (not shown) on the display 114. For example, the workstation 102 may be configured to display an estimate of a patient's blood oxygen saturation generated by the PPG sub-system 104 (referred to as an SpO₂ measurement), and blood pressure from the blood pressure sub-system 106 on the display 114.

The workstation 102 may include any suitable computer-readable media used for data storage. Computer-readable media are configured to store information that may be interpreted by the workstation 102 in general, and by the cardiac output module 112, the PPG analysis module 108, and the blood pressure analysis module 110, in particular. The information may be data or may take the form of computer-executable instructions, such as software applications, that cause the microprocessor to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

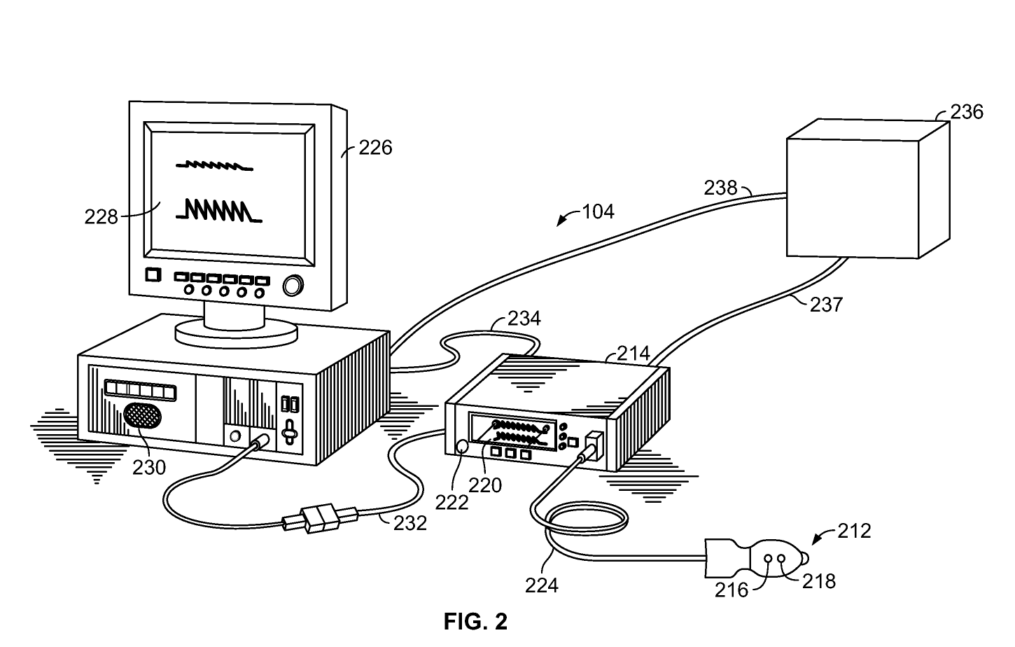

FIG. 2 illustrates an isometric view of the PPG sub-system 104, according to an embodiment. While the sub-system 104 is shown and described as a PPG sub-system, the sub-system 104 may be various other types of physiological detection systems, such as an electrocardiogram system, a phonocardiogram system, and the like. The PPG sub-system 104 may be a pulse oximetry system, for example. The PPG sub-system 104 may include a PPG sensor 212 and a PPG monitor 214. The PPG sensor 212 may include an emitter 216 configured to emit light into tissue of a patient. For example, the emitter 216 may be configured to emit light at two or more wavelengths into the tissue of the patient. The PPG sensor 212 may also include a detector 218 that is configured to detect emitted light from the emitter 216 that emanates from the tissue after passing through the tissue.

The PPG sub-system 104 may include a plurality of sensors forming a sensor array in place of the PPG sensor 212. Each of the sensors of the sensor array may be a complementary metal oxide semiconductor (CMOS) sensor, for example. Alternatively, each sensor of the array may be a charged coupled device (CCD) sensor. In another embodiment, the sensor array may include a combination of CMOS and CCD sensors. The CCD sensor may include a photoactive region and a transmission region configured to receive and transmit, while the CMOS sensor may include an integrated circuit having an array of pixel sensors. Each pixel may include a photodetector and an active amplifier.

The emitter 216 and the detector 218 may be configured to be located at opposite sides of a digit, such as a finger or toe, in which case the light that emanates from the tissue passes completely through the digit. The emitter 216 and the detector 218 may be arranged so that light from the emitter 216 penetrates the tissue and is reflected by the tissue into the detector 218, such as a sensor designed to obtain pulse oximetry data.

The sensor 212 or sensor array may be operatively connected to and draw power from the monitor 214. Optionally, the sensor 212 may be wirelessly connected to the monitor 214 and include a battery or similar power supply (not shown). The monitor 214 may be configured to calculate physiological parameters based at least in part on data received from the sensor 212 relating to light emission and detection. Alternatively, the calculations may be performed by and within the sensor 212 and the result of the oximetry reading may be passed to the monitor 214. Additionally, the monitor 214 may include a display 220 configured to display the physiological parameters and/or other information about the PPG sub-system 104. The monitor 214 may also include a speaker 222 configured to provide an audible sound that may be used in various other embodiments, such as for example, sounding an audible alarm in the event that physiological parameters are outside a predefined normal range.

The sensor 212, or the sensor array, may be communicatively coupled to the monitor 214 via a cable 224. Alternatively, a wireless transmission device (not shown) or the like may be used instead of, or in addition to, the cable 224.

The sub-system 104 may also include a multi-parameter workstation 226 operatively connected to the monitor 214. The workstation 226 may be part of, or the same as, the workstation 102 shown in FIG. 1. Alternatively, the workstation 226 may be in addition to the workstation 102 shown in FIG. 1. The workstation 226 may include a computing sub-system 230, such as standard computer hardware. The computing sub-system 230 may include one or more modules and control units, such as processing devices that may include one or more microprocessors, microcontrollers, integrated circuits, memory, such as read-only and/or random access memory, and the like. The workstation 226 may include a display 228, such as a cathode ray tube display, a flat panel display, such as a liquid crystal display (LCD), light-emitting diode (LED) display, a plasma display, or any other type of monitor. The computing sub-system 230 of the workstation 226 may be configured to calculate physiological parameters and to show information from the monitor 214 and from other medical monitoring devices or systems, such as the blood pressure sub-system 106 (shown in FIG. 1) on the display 228. For example, the workstation 226 may be configured to display an estimate of a patient's blood oxygen saturation generated by the monitor 214 (referred to as an SpO₂ measurement), pulse rate information from the monitor 214 and blood pressure from the blood pressure sub-system 106 (shown in FIG. 1) on the display 228.

The monitor 214 may be communicatively coupled to the workstation 226 via a cable 232 and/or 234 that is coupled to a sensor input port or a digital communications port, respectively and/or may communicate wirelessly with the workstation 226. Additionally, the monitor 214 and/or workstation 226 may be coupled to a network to enable the sharing of information with servers or other workstations. The monitor 214 may be powered by a battery or by a conventional power source such as a wall outlet.

The PPG sub-system 104 may also include a fluid delivery device 236 that is configured to deliver fluid to a patient. The fluid delivery device 236 may be an intravenous line, an infusion pump, any other suitable fluid delivery device, or any combination thereof that is configured to deliver fluid to a patient. The fluid delivered to a patient may be saline, plasma, blood, water, any other fluid suitable for delivery to a patient, or any combination thereof. The fluid delivery device 236 may be configured to adjust the quantity or concentration of fluid delivered to a patient.

The fluid delivery device 236 may be communicatively coupled to the monitor 214 via a cable 237 that is coupled to a digital communications port or may communicate wirelessly with the workstation 226. Alternatively, or additionally, the fluid delivery device 236 may be communicatively coupled to the workstation 226 via a cable 238 that is coupled to a digital communications port or may communicate wirelessly with the workstation 226.

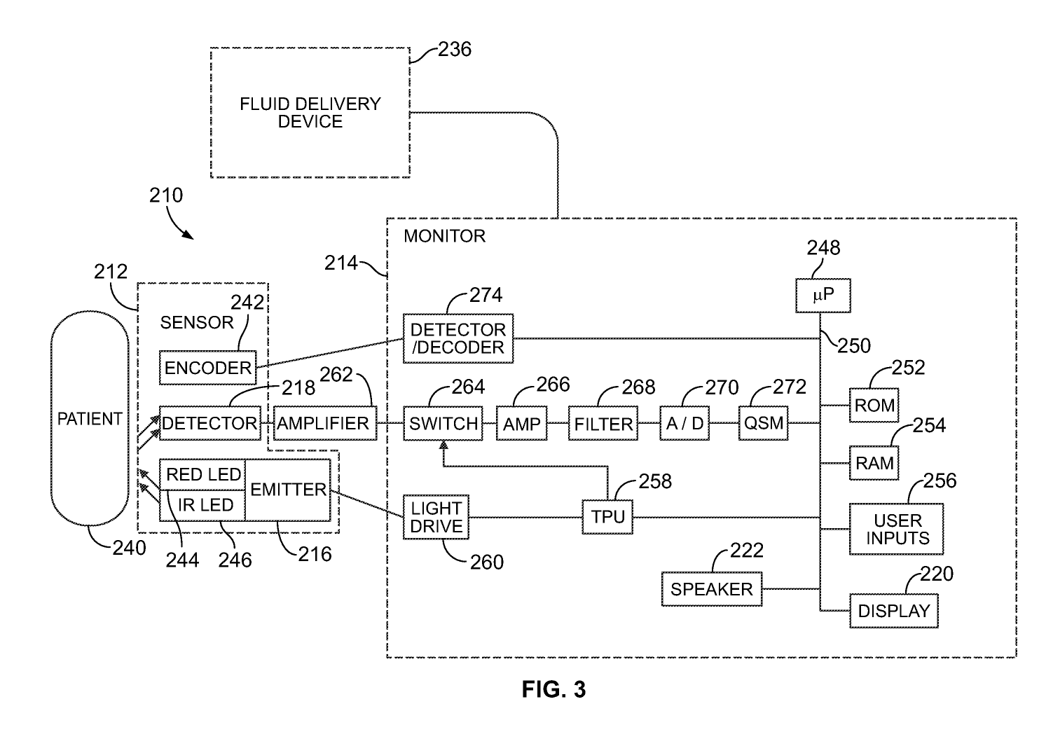

FIG. 3 illustrates a simplified block diagram of the PPG sub-system 104, according to an embodiment. When the PPG sub-system 104 is a pulse oximetry system, the emitter 216 may be configured to emit at least two wavelengths of light (for example, red and infrared) into tissue 240 of a patient. Accordingly, the emitter 216 may include a red light-emitting light source such as a red light-emitting diode (LED) 244 and an infrared light-emitting light source such as an infrared LED 246 for emitting light into the tissue 240 at the wavelengths used to calculate the patient's physiological parameters. For example, the red wavelength may be between about 600 nm and about 700 nm, and the infrared wavelength may be between about 800 nm and about 1000 nm. In embodiments where a sensor array is used in place of single sensor, each sensor may be configured to emit a single wavelength. For example, a first sensor may emit a red light while a second sensor may emit an infrared light.

As discussed above, the PPG sub-system 104 is described in terms of a pulse oximetry system. However, the PPG sub-system 104 may be various other types of systems. For example, the PPG sub-system 104 may be configured to emit more or less than two wavelengths of light into the tissue 240 of the patient. Further, the PPG sub-system 104 may be configured to emit wavelengths of light other than red and infrared into the tissue 240. As used herein, the term “light” may refer to energy produced by radiative sources and may include one or more of ultrasound, radio, microwave, millimeter wave, infrared, visible, ultraviolet, gamma ray or X-ray electromagnetic radiation. The light may also include any wavelength within the radio, microwave, infrared, visible, ultraviolet, or X-ray spectra, and that any suitable wavelength of electromagnetic radiation may be used with the PPG sub-system 104. The detector 218 may be configured to be specifically sensitive to the chosen targeted energy spectrum of the emitter 216.

The detector 218 may be configured to detect the intensity of light at the red and infrared wavelengths. Alternatively, each sensor in the array may be configured to detect an intensity of a single wavelength. In operation, light may enter the detector 218 after passing through the tissue 240. The detector 218 may convert the intensity of the received light into an electrical signal. The light intensity may be directly related to the absorbance and/or reflectance of light in the tissue 240. For example, when more light at a certain wavelength is absorbed or reflected, less light of that wavelength is received from the tissue by the detector 218. After converting the received light to an electrical signal, the detector 218 may send the signal to the monitor 214, which calculates physiological parameters based on the absorption of the red and infrared wavelengths in the tissue 240.

In an embodiment, an encoder 242 may store information about the sensor 212, such as sensor type (for example, whether the sensor is intended for placement on a forehead or digit) and the wavelengths of light emitted by the emitter 216. The stored information may be used by the monitor 214 to select appropriate algorithms, lookup tables and/or calibration coefficients stored in the monitor 214 for calculating physiological parameters of a patient. The encoder 242 may store or otherwise contain information specific to a patient, such as, for example, the patient's age, weight, and diagnosis. The information may allow the monitor 214 to determine, for example, patient-specific threshold ranges related to the patient's physiological parameter measurements, and to enable or disable additional physiological parameter algorithms. The encoder 242 may, for instance, be a coded resistor that stores values corresponding to the type of sensor 212 or the types of each sensor in the sensor array, the wavelengths of light emitted by emitter 216 on each sensor of the sensor array, and/or the patient's characteristics. Optionally, the encoder 242 may include a memory in which one or more of the following may be stored for communication to the monitor 214: the type of the sensor 212, the wavelengths of light emitted by emitter 216, the particular wavelength each sensor in the sensor array is monitoring, a signal threshold for each sensor in the sensor array, any other suitable information, or any combination thereof.

Signals from the detector 218 and the encoder 242 may be transmitted to the monitor 214. The monitor 214 may include a general-purpose control unit, such as a microprocessor 248 connected to an internal bus 250. The microprocessor 248 may be configured to execute software, which may include an operating system and one or more applications, as part of performing the functions described herein. A read-only memory (ROM) 252, a random access memory (RAM) 254, user inputs 256, the display 220, and the speaker 222 may also be operatively connected to the bus 250.

The RAM 254 and the ROM 252 are illustrated by way of example, and not limitation. Any suitable computer-readable media may be used in the system for data storage. Computer-readable media are configured to store information that may be interpreted by the microprocessor 248. The information may be data or may take the form of computer-executable instructions, such as software applications, that cause the microprocessor to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

The monitor 214 may also include a time processing unit (TPU) 258 configured to provide timing control signals to a light drive circuitry 260, which may control when the emitter 216 is illuminated and multiplexed timing for the red LED 244 and the infrared LED 246. The TPU 258 may also control the gating-in of signals from the detector 218 through an amplifier 262 and a switching circuit 264. The signals are sampled at the proper time, depending upon which light source is illuminated. The received signal from the detector 218 may be passed through an amplifier 266, a low pass filter 268, and an analog-to-digital converter 270. The digital data may then be stored in a queued serial module (QSM) 272 (or buffer) for later downloading to RAM 254 as QSM 272 fills up. In an embodiment, there may be multiple separate parallel paths having amplifier 266, filter 268, and A/D converter 270 for multiple light wavelengths or spectra received.

The microprocessor 248 may be configured to determine the patient's physiological parameters, such as SpO₂ and pulse rate, using various algorithms and/or look-up tables based on the value(s) of the received signals and/or data corresponding to the light received by the detector 218. The signals corresponding to information about a patient, and regarding the intensity of light emanating from the tissue 240 over time, may be transmitted from the encoder 242 to a decoder 274. The transmitted signals may include, for example, encoded information relating to patient characteristics. The decoder 274 may translate the signals to enable the microprocessor 248 to determine the thresholds based on algorithms or look-up tables stored in the ROM 252. The user inputs 256 may be used to enter information about the patient, such as age, weight, height, diagnosis, medications, treatments, and so forth. The display 220 may show a list of values that may generally apply to the patient, such as, for example, age ranges or medication families, which the user may select using the user inputs 256.

The fluid delivery device 236 may be communicatively coupled to the monitor 214. The microprocessor 248 may determine the patient's physiological parameters, such as a change or level of fluid responsiveness, and display the parameters on the display 220. In an embodiment, the parameters determined by the microprocessor 248 or otherwise by the monitor 214 may be used to adjust the fluid delivered to the patient via the fluid delivery device 236.

As noted, the PPG sub-system 104 may be a pulse oximetry system. A pulse oximeter is a medical device that may determine oxygen saturation of blood. The pulse oximeter may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of a patient. Pulse oximeters typically measure and display various blood flow characteristics including, but not limited to, the oxygen saturation of hemoglobin in arterial blood.

A pulse oximeter may include a light sensor, similar to the sensor 212, that is placed at a site on a patient, typically a fingertip, toe, forehead or earlobe, or in the case of a neonate, across a foot. The pulse oximeter may pass light using a light source through blood perfused tissue and photoelectrically sense the absorption of light in the tissue. For example, the pulse oximeter may measure the intensity of light that is received at the light sensor as a function of time. A signal representing light intensity versus time or a mathematical manipulation of this signal (for example, a scaled version thereof, a log taken thereof, a scaled version of a log taken thereof, and/or the like) may be referred to as the photoplethysmograph (PPG) signal. In addition, the term “PPG signal,” as used herein, may also refer to an absorption signal (for example, representing the amount of light absorbed by the tissue) or any suitable mathematical manipulation thereof. The light intensity or the amount of light absorbed may then be used to calculate the amount of the blood constituent (for example, oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with lower oxygen saturation. By comparing the intensities of two wavelengths at different points in the pulse cycle, it is possible to estimate the blood oxygen saturation of hemoglobin in arterial blood.

The PPG sub-system 104 and pulse oximetry are further described in United States Patent Application Publication No. 2012/0053433, entitled “System and Method to Determine SpO₂ Variability and Additional Physiological Parameters to Detect Patient Status,” United States Patent Application Publication No. 2012/0029320, entitled “Systems and Methods for Processing Multiple Physiological Signals,” United States Patent Application Publication No. 2010/0324827, entitled “Fluid Responsiveness Measure,” and United States Patent Application Publication No. 2009/0326353, entitled “Processing and Detecting Baseline Changes in Signals,” all of which are hereby incorporated by reference in their entireties.

Respiratory variation of the PPG signal may correlate with fluid responsiveness. Fluid responsiveness relates to the volume of fluid, such as blood, in the arteries, veins, and vasculature of an individual. In general, fluid responsiveness may include a measurement of the response of stroke volume, the volume of blood passing out of the heart with each heartbeat, to venous return, the volume of blood entering the heart with each heartbeat, caused by the clinical administration of fluid into the vasculature, such as through an intravenous injection. With each heartbeat, a certain amount of blood is pumped out of the heart. The more blood that fills the heart, the more blood the heart can pump out with each heartbeat. If blood volume is too low, the heart may not fully fill with blood. Therefore, the heart may not pump out as much blood with each heartbeat. Consequently, low blood volume may lead to low blood pressure, and organs and tissues may not receive enough blood to optimally and/or properly function. Monitoring fluid responsiveness allows a physician to determine whether additional fluid should be provided to a patient, such as through an intravenous fluid injection. In short fluid responsiveness represents a prediction of whether or not additional intravenous fluid may improve blood flow within a patient. Fluid responsiveness may be viewed as a response of a heart in relation to overall fluid within a patient.

Fluid responsiveness may be monitored in, for example, critically-ill patients because fluid administration plays an important role in optimizing stroke volume, cardiac output, and oxygen delivery to organs and tissues. However, clinicians need to balance central blood volume depletion and volume overloading. Critically-ill patients are generally at greater risk for volume depletion and severe hypotension is a common life-threatening condition in critically-ill patients. Conversely, administering too much fluid may induce life-threatening adverse effects, such as volume overload, systemic and pulmonary edema, and increased tissue hypoxia. Therefore, obtaining reliable information and parameters that aid clinicians in fluid management decisions may help improve patient outcomes.

FIG. 4 illustrates a PPG signal 400 over time, according to an embodiment. The PPG signal 400 is an example of a physiological signal. However, embodiments may be used in relation to various other physiological signals, such as electrocardiogram signals, phonocardiogram signals, ultrasound signals, and the like. The PPG signal 400 may be determined, formed, and displayed as a waveform by the monitor 214 (shown in FIG. 2) that receives signal data from the PPG sensor 212 (shown in FIG. 2). For example, the monitor 214 may receive signals from the PPG sensor 212 positioned on a finger of a patient. The monitor 214 processes the received signals, and displays the resulting PPG signal 400 on the display 228 (shown in FIG. 2), and/or the display 114 (shown in FIG. 1).

The PPG signal 400 may be generated by the PPG sub-system 104. The workstation 102 may receive the PPG signal 400 from the PPG sub-system 104. The PPG signal 400 may be analyzed by the microprocessor 248 of the PPG sub-system 104, and/or the PPG signal 400 may be analyzed by the PPG analysis module 108 (shown in FIG. 1) of the workstation 102.

The PPG signal 400 may include a plurality of pulses 402a-402n over a predetermined time period. The time period may be a fixed time period, or the time period may be variable. Moreover, the time period may be a rolling time period, such as a 5 second rolling timeframe.

Each pulse 402a-402n may represent a single heartbeat and may include a pulse-transmitted or primary peak 404 separated from a pulse-reflected or trailing peak 406 by a dichrotic notch 408. The primary peak 404 represents a pressure wave generated from the heart to the point of detection, such as in a finger where the PPG sensor 212 (shown in FIG. 2) is positioned. The trailing peak 406 may represent a pressure wave that is reflected from the location proximate where the PPG sensor 212 is positioned back toward the heart.

As shown in FIG. 4, each pulse 402a-402n has a particular amplitude. For example, the pulse 402a has an amplitude a₁, while the pulse 402b has an amplitude a₂. The amplitudes a₁ and a₂ may differ, as shown. Indeed, the amplitudes of each pulse 402a-402n may vary with respect to one another. In general, the overall amplitude of the pulse in the PPG signal 400 over time t may modulate. As a side note, the PPG signal has various other aspects, parameters, or the like that may modulate over time. For example, signals related to respiration, blood pressure, vasomotion, and the like may modulate over time. Embodiments of the present disclosure may analyze modulations of the pulse component of the PPG signal 400, and/or various other components, parameters, aspects of the PPG signal 400. The PPG analysis module 108 of the workstation 102 (shown in FIG. 1) may track and store the magnitude of the amplitude modulation of the pulse of the PPG signal 400 over time t. Optionally, the PPG analysis module 108 may track and store the magnitude of the amplitude modulation of the PPG signal 400 over time periods of varying lengths. For example, the PPG analysis module 108 may compare the amplitudes of neighboring pulses 402a and 402b and store the change in amplitude between the two pulses 402a and 402b as a magnitude of amplitude modulation. The PPG analysis module 108 may continually track and store amplitude modulation between neighboring pulses 402a-402n over the time period t. Optionally, the PPG analysis module 108 may determine an indication of the strength of the modulation of the pulses 402a-402n over the time period t, which may be or include an average modulation, or a difference between a maximum and minimum pulse amplitude, or some other measures of amplitude modulation. The strength of the modulation may be stored as a magnitude of amplitude modulation. Alternatively, the PPG analysis module 108 may determine an amplitude change of a PPG signal by directly comparing PPG waveforms. For example, a PPG waveform may be superimposed over a second PPG waveform, and the PPG analysis module 108 may determine a change therebetween through the difference in waveform shapes.

The PPG signal 400 also includes a baseline 410, which may modulate over the time period t. The PPG analysis module 108 may monitor and store the baseline modulation Δb over the time period t. Optionally, the PPG analysis module 108 may determine the baseline modulation between neighboring peaks 402a-402n. As shown in FIG. 4, however, the magnitude of baseline modulation Δb may be summed, averaged, or otherwise determined over the time period t.

As described, the PPG analysis module 108 may track, store, and analyze the PPG signal 400. Alternatively, the monitor 214 of the PPG sub-system 104 (shown in FIG. 2) may include one or more modules to track, store, and analyze the PPG signal 400. In such an embodiment, the workstation 102 may receive data related to the PPG signal 400 from the PPG sub-system 104. For example, the PPG sub-system 104 may include the PPG analysis module 108.

It has been found that amplitude modulation relates to stroke volume. For example, amplitude modulation may be directly proportional to stroke volume. The higher the magnitude of amplitude modulation, the higher the stroke volume.

Similarly, it has been found that baseline modulation relates to venous return. For example, baseline modulation may be directly proportional to venous return. The higher the magnitude of baseline modulation, the higher the venous return.

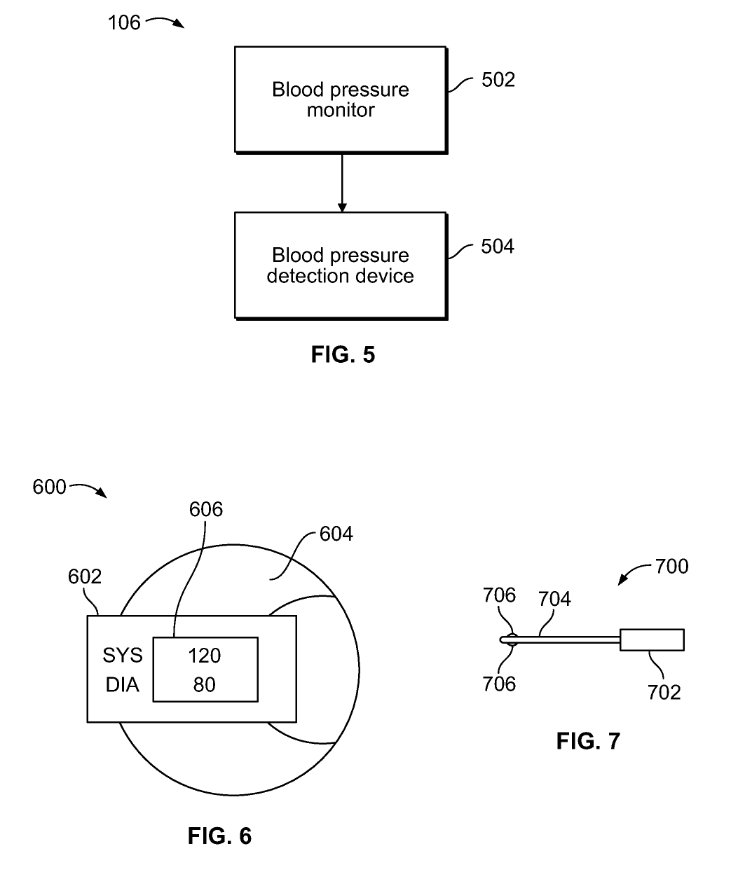

FIG. 5 illustrates a simplified block diagram of the blood pressure sub-system 106, according to an embodiment. The blood pressure sub-system 106 may include a blood pressure monitor 502 operatively connected to a blood pressure detection device 504.

FIG. 6 illustrates a simplified front view of a blood pressure sub-system 600, according to an embodiment. The blood pressure sub-system 106 may include a blood pressure monitor 602 operatively connected to a blood pressure cuff 604 configured to be positioned around a portion of an arm of a patient. As shown in FIG. 6, the blood pressure sub-system 106 may allow for non-invasive blood pressure detection by way of the cuff 604 being positioned around a portion of patient anatomy. The blood pressure monitor 602 may include a digital blood pressure monitor having a display 606 that shows blood pressure data. The blood pressure monitor 602 may be in communication with the blood pressure analysis module 110 (shown in FIG. 1) of the system 100. Alternatively, the blood pressure monitor 602 may include the blood pressure analysis module 110. Also, alternatively, the blood pressure sub-system 106 may include a sphygmomanometer and stethoscope used by an individual to detect blood pressure. Blood pressure data may then be directly input into the workstation 102, such as through a keyboard, mouse, or the like, and analyzed by the blood pressure analysis module 110.

FIG. 7 illustrates a simplified lateral view of a blood pressure detection device 700, according to an embodiment. The blood pressure detection device 700 may include a housing 702 connected to a catheter 704 configured to be positioned within vasculature of a patient. The catheter 704 may include one or more pressure-detection sensors 706, such as piezoelectric transducers, at various points along its length. The pressure-detection sensors 706 are configured to detect pressure pulses of blood within the vasculature. The blood pressure detection device 700 may be, for example, an arterial line (A-line) catheter. The blood pressure detection device 700 may be operatively connected to and in communication with the blood pressure monitor 502 (shown in FIG. 5).

The blood pressure detection device 700 may include a thin catheter configured to be inserted into an artery of a patient. Accordingly, the blood pressure detection device 700 may be used to detect blood pressure in real time, rather than through intermittent measurement. The blood pressure detection device 700 may be inserted into the radial artery proximate the wrist, the brachial artery proximate the elbow, the femoral artery proximate the groin, the dorsalis pedis artery proximate the foot, or into the ulnar artery inside the wrist, for example. However, the blood pressure detection device 700 may be configured to be positioned within various other arteries, veins, and vasculature at various other portions of a patient's body.

Referring to FIGS. 5-7, the blood pressure sub-system 106 may be any type of system configured to detect and monitor blood pressure. FIGS. 6 and 7 merely provide examples of such a sub-system 106, monitor 502, and detection device 504. The blood pressure detection device 504 may be an invasive, non-invasive, or minimally invasive device configured to detect blood pressure. For example, the blood pressure detection device 504 may be various types of invasive, non-invasive tonometric and volume clamping systems, as well as auscultation, oscillometric and other such devices.

The blood pressure monitor 502 may be configured to calculate blood pressure of an individual based at least in part on data received from the blood pressure detection device 504. The blood pressure monitor 502 may include a display, such as the display 606, configured to display the blood pressure. The blood pressure detection device 504 may be communicatively coupled to the blood pressure monitor 502 via a cable, wireless connection, or the like. The blood pressure monitor 502 may include one or more modules and control units, such as processing devices that may include one or more microprocessors, microcontrollers, integrated circuits, memory, such as read-only and/or random access memory, and the like. Accordingly, the blood pressure monitor may be configured to calculate blood pressure and to show information related to blood pressure on a display. The blood pressure monitor 502 may be communicatively coupled to the workstation 102 (shown in FIG. 1) via a cable that is coupled to a sensor input port or a digital communications port, respectively and/or may communicate wirelessly with the workstation 102. Additionally, the blood pressure monitor 502 may be coupled to a network to enable the sharing of information with servers or other workstations. The blood pressure monitor 502 may be powered by a battery or by a conventional power source such as a wall outlet.

The blood pressure sub-system 106 is configured to detect the pressure exerted by circulating blood within vasculature of an individual. During each heartbeat, blood pressure varies between a maximum (systolic) and a minimum (diastolic) pressure. In general, a blood pressure may relate to cardiac output and physiological resistance to blood flow, as shown below:

P=CO×R Equation (1)

where P is blood pressure that drives the blood flow through the vascular system, CO is cardiac output, and R is a physiological resistance that impedes the flow of blood. The physiological resistance may be a vascular resistance, for example. Thus, referring to Equation (1), an abnormal change in blood pressure may indicate problems with cardiac output, blood vessel resistance, or both.

The blood pressure may be or include a mean arterial pressure (MAP). MAP may be determined by the cardiac output (CO), systemic vascular resistance (SVR), and central venous pressure (CVP), as shown below:

MAP=(CO×SVR)+CVP Equation (2)

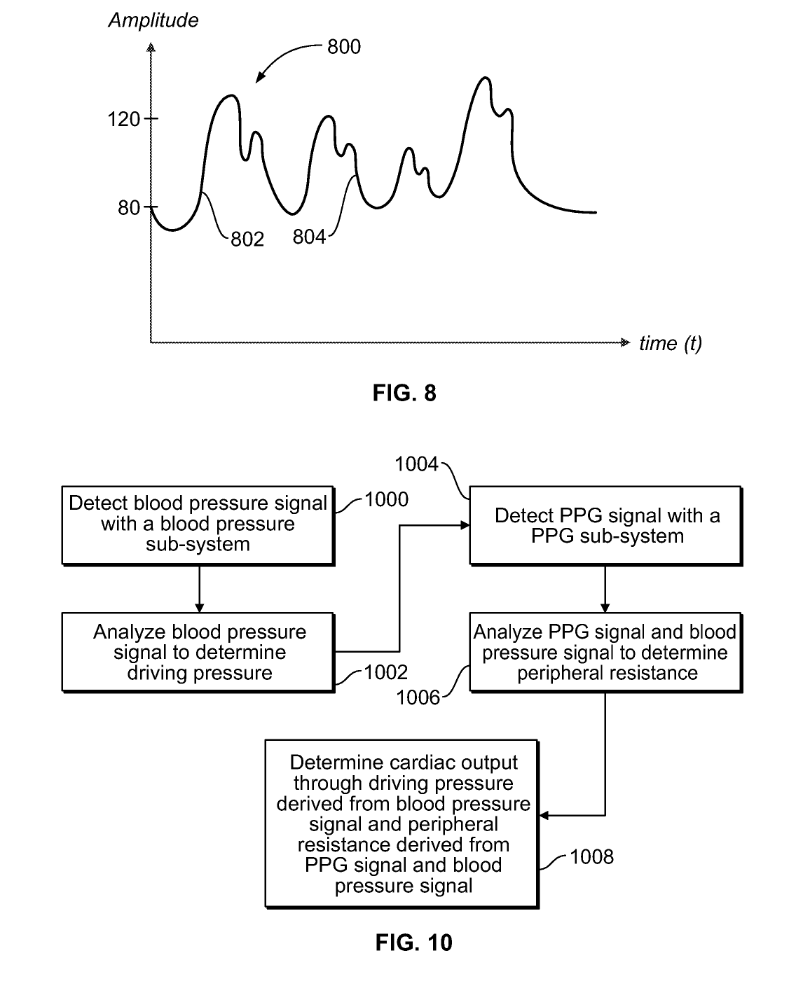

FIG. 8 illustrates a blood pressure signal 800 over time, according to an embodiment. The blood pressure signal 800, like the PPG signal 400 (shown in FIG. 4), is an example of a physiological signal. As shown, a physiological parameter, such as an amplitude of the blood pressure signal 800, may vary over time. For example, the amplitude may vary with respect to a base, average, or mean blood pressure of 120 systolic over 80 diastolic. As an example, the amplitude may change from blood pressure pulse 802 to blood pressure pulse 804. The blood pressure analysis module 110 (shown in FIG. 1), which may be part of the workstation 102, or the blood pressure sub-system 106, may track the change in amplitude of the blood pressure signal 800, and store the change in amplitude for analysis. The blood pressure analysis module 110 may track and store amplitude changes between neighboring blood pressure pulses, such as pulses 802 and 804. Alternatively, the blood pressure analysis module 110 may determine an average amplitude modulation over a particular time frame, for example. Also, alternatively, the blood pressure analysis module 110 may determine an amplitude change of a blood pressure signal by directly comparing blood pressure waveforms. For example, a first blood pressure waveform may be superimposed over a second blood pressure waveform, and the blood pressure analysis module 110 may determine a change between blood pressure waveforms through the difference in waveform shapes. For example, an amplitude change between the first and second blood pressure waveforms may be a difference between a maximum and minimum amplitude over a respiratory cycle. The amplitude change may represent an average of the difference between the maximum and minimum amplitudes of the blood pressure waveforms over a number of respiratory cycles.

Referring again to FIG. 1, the cardiac output determination module 112 receives data signals from the PPG sub-system 104 and the blood pressure sub-system 106. The PPG sub-system 104 may generate a PPG signal (such as shown in FIG. 4) that is received by the PPG analysis module 108, which then analyzes the PPG signal, such as the PPG waveform, to determine amplitude modulation of the PPG signal, for example. Optionally, the PPG signal may be analyzed to determine baseline modulation and/or frequency modulation, in addition to, or instead of, amplitude modulation. Similarly, the blood pressure sub-system 106 may generate a blood pressure signal (such as shown in FIG. 8) that is received by the blood pressure analysis module 110, which may then analyze the blood pressure signal, such as a blood pressure waveform, to determine amplitude modulation of the blood pressure signal. Optionally, the blood pressure signal may be analyzed to determine baseline modulation and/or frequency modulation, in addition to, or instead of, amplitude modulation. The cardiac output determination module 112 then receives the data from the PPG analysis module 108 and the blood pressure analysis module 110 to determine cardiac output, based on data received from the PPG sub-system 104 and the blood pressure sub-system 106. As noted, the blood pressure analysis module 110 and the PPG analysis module 108 may be part of the workstation 102. Alternatively, the blood pressure analysis module 110 may be part of the blood pressure sub-system 106, while the PPG analysis module 108 may be part of the PPG subsystem 104.

The blood pressure analysis module 110 and/or the cardiac output determination module 112 may use a blood pressure parameter, such as a mean arterial pressure, systolic pressure, diastolic pressure, pulse pressure (which may be derived from the systolic and diastolic pressures, for example) or the like, as an indication of driving pressure of the circulatory system. For example, the blood pressure analysis module 110 and/or the cardiac output determination module 112 may be used to determine the driving pressure, which may be or include the mean arterial pressure, systolic blood pressure, diastolic blood pressure, and/or any combination thereof. Further, the PPG analysis module 108 and/or the cardiac output determination module 112 may use a change or modulation in amplitude of the PPG signal 400 (shown in FIG. 4) to determine peripheral vascular resistance, which represents a resistance to the flow of blood determined by the tone of the vascular musculature and the diameter of the blood vessels. Also, the PPG analysis module 108 may determine peripheral vascular resistance in combination with the blood pressure analysis module 110. For example, the PPG analysis module 108 may use a change or modulation in amplitude of the PPG signal with respect to an associated change or modulation in amplitude of the blood pressure signal to determine peripheral vascular resistance.

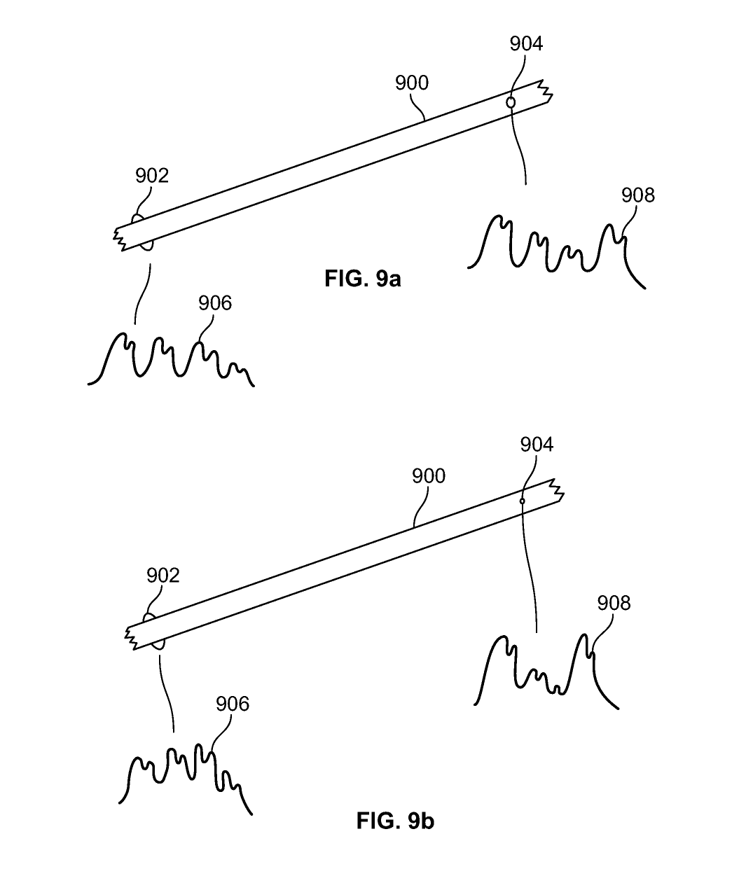

FIGS. 9a and 9b illustrate simplified views of a blood vessel 900 with a PPG detector 902 and a blood pressure detection device 904 detecting PPG signals 906 and blood pressure signals 908, respectively, at first time and second times, respectively, according to an embodiment of the present disclosure. It is to be understood that the vessel 900 may be more than one vessel throughout a body of a patient. For example, the PPG detector 902 may be proximate a finger of a patient, while the blood pressure detection device 904 may between an elbow and shoulder of the patient. The PPG detector 902 may be any of the PPG detectors 902 discussed above, while the blood pressure detection device 904 may be any of the blood pressure detection devices discussed above.

In order to calculate cardiac output, a driving pressure and a resistance are detected, as shown in Equation (1) above. The driving pressure may be detected from the blood pressure signal, which is detected by the blood pressure detection device 904. For example, the driving pressure may be MAP. In order to determine the resistance to flow, amplitude modulations in the blood pressure signal 908 and the PPG signal 906 are analyzed. By analyzing both the amplitude modulations in the blood pressure signal 908 and the resulting modulations in the PPG signal 906 over time, such as between the first time as represented in FIG. 9a to the second time as represented in FIG. 9b, the cardiac output determination module 112, for example, can determine how the PPG signal 906 responds for a given blood pressure signal 908.

For example, if the blood pressure signal 908 exhibits a large amplitude modulation and the PPG signal 906 also exhibits a large amplitude modulation, then it may be determined that the resistance at the peripheries of the vessel 900 has decreased, as the vessel 900 is relatively compliant. If, however, there is a large amplitude modulation in the blood pressure signal 908, but a correspondingly low amplitude modulation in the PPG signal 906, it may be determined that there is a higher resistance at the peripheries, or that the vessel 900 has become less compliant. Therefore, in order to determine vascular resistance, the system and method of the present disclosure may analyze the behavior of the PPG signal 906 for a given modulation of a blood pressure signal 908 in order to determine resistance.

Alternatively, if an amplitude of the blood pressure signal 908 exhibits a high degree of modulation, variation, or other such change (for example, a high degree of variation from a mean blood pressure signal, between blood pressure pulses, and/or with respect to an averaged blood pressure signal), then the cardiac output determination module 112 may determine that the driving pressure correlates with a small degree of peripheral vascular resistance. Conversely, if an amplitude of the blood pressure signal 908 exhibits a low degree of modulation, variation, or other such change, then the cardiac output determination module 112 may determine that the driving pressure correlates with a high degree of peripheral vascular resistance. Thus, the change in the amplitude of the blood pressure signal (Aₚᵣₑₛₛ) may be inversely proportional to peripheral vascular resistance.

Further, amplitude modulation of the PPG signal 906 may be directly proportional to stroke volume. The higher the magnitude of amplitude modulation, the higher the stroke volume. Also, for example, baseline modulation of the PPG signal 906 may be directly proportional to venous return. The higher the magnitude of baseline modulation, the higher the venous return.

It has been found that an increase in a blood pressure parameter, such as MAP, systolic pressure, diastolic pressure, and/or the like, may indicate a greater driving pressure for the circulatory system, and, therefore, an increase in cardiac output. Further, an increase in a PPG parameter, such as amplitude, baseline, frequency, or the like, may indicate higher arterial compliance, which may be indicative of lower peripheral resistance for the circulatory system which, for a given blood pressure, may lead to an increase in cardiac output. Further, when considering this increase in the PPG parameter, inspection of the pulse signal of the blood pressure waveform may provide additional information relevant to a derivation of arterial compliance. For example, a change in a ratio of the PPG pulse amplitude modulation to blood pressure pulse amplitude modulation may indicate a change in arterial vessel compliance. Accordingly, combining an analysis of a blood pressure signal with an analysis of a PPG signal may provide a determination of cardiac output that is more accurate and reliable than an analysis of only one of a blood pressure signal or a PPG signal.

The cardiac output determination module 112 may determine a change in cardiac output by comparing a blood pressure parameter with respect to a PPG parameter. For example, the cardiac output determination module 112 may generate a cardiac output index value that is based on a change in the amplitude of the blood pressure signal modulations with respect to a change in the amplitude of the PPG signal modulations. Cardiac output may be a function of a change in the blood pressure parameter with respect to a change in the PPG parameter.

As an example, a blood pressure amplitude measurement (Aₚᵣₑₛₛ) may be determined. Aₚᵣₑₛₛ may change a certain percentage over time. Thus, ΔAₚᵣₑₛₛ may simply be a scalar quality, such as 0.2 (which would indicate a 20% change over a predefined time). Similarly, a PPG amplitude (Aₚₗₑₜₕ) may be determined. Aₚₗₑₜₕ may change a certain percentage over time. Thus, ΔAₚₗₑₜₕ may also simply be a scalar quality, such as 0.2 (which would indicate a 20% change over a predefined time). A change in resistance to blood flow R may therefore be dependent on a change in amplitude strength of the PPG signal modulation relative to the change in amplitude strength of the blood pressure modulation. Therefore, a change in resistance may be determined by a change in Aₚₗₑₜₕ with respect to a change in Aₚᵣₑₛₛ, using a formula combining both parameters, as shown below.

ΔR=Δ(ΔAₚₗₑₜₕ/ΔAₚᵣₑₛₛ) Equation (3), or

ΔR=Δ(ΔAₚᵣₑₛₛ/ΔAₚₗₑₜₕ) Equation (4)

where ΔR is a change in the resistance to blood flow, ΔAₚₗₑₜₕ is a change in amplitude of a PPG signal, and ΔAₚᵣₑₛₛ is a change in amplitude of a blood pressure signal. Instead of the resistance to blood flow equaling the ratios noted above, the resistance to blood flow may simply be a function of the ratios noted above. As shown, the cardiac output determination module 112 may determine a change in cardiac output (ΔCO) by using the information concerning the change in resistance to blood flow (for example, by determining a change in ratio ΔAₚₗₑₜₕ/ΔAₚᵣₑₛₛ). Thus, if the ratio between ΔAₚₗₑₜₕ/ΔAₚᵣₑₛₛ is the same, and MAP remains the same, then the cardiac output determination module 112 determines that the cardiac output has not changed. If, however, there is a large discrepancy between the ΔAₚₗₑₜₕ/ΔAₚᵣₑₛₛ, for example, then the cardiac output determination module 112 may determine a higher degree of change in R and hence cardiac output. Accordingly, ΔR may be directly proportional to the ration of ΔAₚₗₑₜₕ to ΔAₚᵣₑₛₛ. The relative amplitude modulations of the PPG signal and the blood pressure signal provide information that may yield a calculation of R, which may then be used to determine CO.

In an embodiment, the resistance may be a function of ΔAₚₗₑₜₕ and ΔAₚᵣₑₛₛ. That is, R=f(ΔAₚₗₑₜₕ, ΔAₚᵣₑₛₛ). For example, referring to equations (3) and (4), the linear relationship may be as follows:

R=m(ΔAₚₗₑₜₕ/ΔAₚᵣₑₛₛ)+c Equation (5)

R=m(ΔAₚᵣₑₛₛ/ΔAₚₗₑₜₕ)+c Equation (6)

where m is a gradient and c is a constant value. The relationships may be derived from best fit lines to historical data sets.

Accordingly, even though a blood pressure signal 800 may be constant, for example, the cardiac output may vary. Analysis of the PPG signal 400 provides enhanced data that more accurately reflects cardiac output in relation to a blood pressure signal 800, even when the blood pressure signal does not appreciably vary over time.

The cardiac output determination module 112 may utilize the ratio ΔAₚᵣₑₛₛ/ΔAₚₗₑₜₕ or ΔAₚₗₑₜₕ/ΔAₚᵣₑₛₛ to determine a change in resistance (R). From Equation (1), Cardiac Output (CO) relates to driving pressure (P) and fluid resistance (R) as follows:

CO=P/R Equation (7)

Accordingly, R may be a function of ΔAₚᵣₑₛₛ/ΔAₚₗₑₜₕ or ΔAₚₗₑₜₕ/ΔAₚᵣₑₛₛ, and P may be obtained directly from the blood pressure signal (for example, P may be the mean arterial pressure). Therefore,

CO=P/f(ΔAₚᵣₑₛₛ,ΔAₚₗₑₜₕ) Equation (8)

where P is the driving pressure, as detected by the blood pressure sub-system 106, and ΔAₚᵣₑₛₛ and ΔAₚₗₑₜₕ may be determined as discussed above. The peripheral resistance, R, is a function of ΔAₚᵣₑₛₛ and ΔAₚₗₑₜₕ, and may be of the form ΔAₚᵣₑₛₛ/ΔAₚₗₑₜₕ and/or ΔAₚₗₑₜₕ/ΔAₚᵣₑₛₛ. The cardiac output determination module 112 tracks and stores the ratio ΔAₚᵣₑₛₛ/ΔAₚₗₑₜₕ and/or ΔAₚₗₑₜₕ/ΔAₚᵣₑₛₛ. The ratio of the change in a PPG parameter, such as PPG amplitude, with respect to the change in a blood pressure parameter, such as blood pressure amplitude, may provide a clearer, more accurate, and more reliable indication of the tracking and trending of cardiac output then an analysis of blood pressure modulation or PPG modulation by themselves.

Further, The cardiac output determination module 112 may utilize the driving pressure P detected by the blood pressure sub-system 106, and the resistance determine by analysis of the PPG signal generated by the PPG sub-system 104 to determine cardiac output. In this embodiment, the cardiac output determination module 112 may not rely on estimates, models, or the like. Instead, the cardiac output determination module 112 may accurately determine cardiac output through actual blood pressure and PPG signals.

Referring to FIGS. 1, 4, and 8, for example, the cardiac output determination module 112 may map the blood pressure signal 800 to the PPG signal 400, or vice versa. The cardiac output determination module 112 may utilize a transfer function to correlate a change in the pressure signal 800 to a change in the PPG signal, or vice versa. The transfer function may be fitted, for example, to a Windkessel model of the peripheral vascular system in order to estimate changes in peripheral compliance and resistance. The estimates may then be used, in conjunction with known mean arterial pressure, for example, to derive cardiac output, or determine changes in cardiac output. For example, a ratio of blood pressure modulation to PPG signal modulation may indicate a pressure dependency of volumetric change. Changes in the ratio may indicate changes in peripheral compliance, which may be correlated with changes in peripheral and systemic vascular resistance. Cardiac output may be derived from these changes and a detection of mean arterial pressure.

FIG. 10 illustrates a flow chart of a method of determining cardiac output, according to an embodiment. At 1000, a blood pressure signal is detected with a blood pressure sub-system. Then, at 1002, the blood pressure signal is analyzed to determine a driving pressure (P).

At 1004, a PPG signal is detected with a PPG sub-system. Then, at 1006, the PPG signal and the blood pressure signal area analyzed to determine peripheral resistance (R). 1004 may be performed simultaneously with 1000. Optionally, 1004 may be performed before or after 1000, or vice versa. Also, alternatively, 1006 may be performed using information from 1002.

After the driving pressure P and the peripheral resistance R are determined, the method proceeds to 1008, in which cardiac output, or a change in cardiac output, is determined through P, which was derived from the blood pressure signal, and R, which was derived from the PPG signal and the blood pressure signal.

Thus, embodiments provide an efficient system and method for quickly and accurately determining cardiac output.

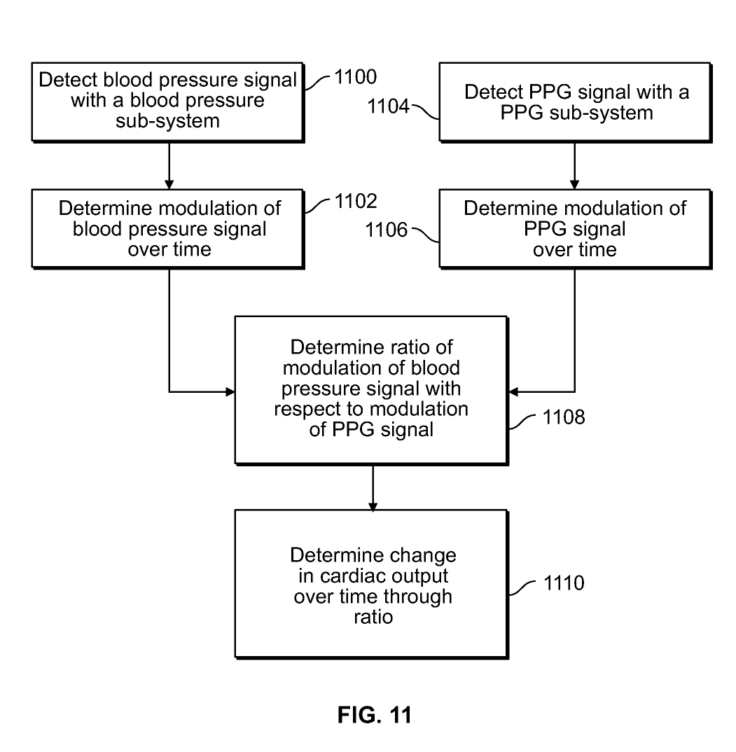

FIG. 11 illustrates a flow chart of a method of determining changes in cardiac output over time, according to an embodiment. At 1100, a blood pressure signal is detected with a blood pressure sub-system. Then, at 1102, a modulation of the blood pressure signal over time is determined. The blood pressure signal modulation may be related to amplitude, baseline, frequency, or the like, of the blood pressure signal. The modulation may be determined as a scalar value. For example, the modulation may be a percentage change over time. The modulation may be determined on a pulse-to-pulse basis, as an average change with respect to a mean value, as an average change over time, as a waveform shape difference determination, and/or the like.

At 1104, a PPG signal is detected with a PPG sub-system. Then, at 1106, a modulation of the PPG signal over time is determined. The modulation of the PPG signal may be with respect to amplitude, baseline, frequency, and/or the like, of the PPG signal. The modulation may be determined as a scalar value. For example, the modulation may be a percentage change over time. The modulation may be determined on a pulse-to-pulse basis, as an average change with respect to a mean value, as an average change over time, as a waveform shape difference determination, and/or the like. 1104 and 1106 may occur simultaneously along with 1100 and 1102, respectively. Optionally, 1104 and 1106 may occur before or after 1100 and 1102, respectively, or vice versa.

After the modulation of the blood pressure signal and the modulation of the PPG signal have been determined, the method continues to 1108, in which a ratio of the blood pressure modulation with respect to the PPG modulation is determined. The ratio may be a function of changes in peripheral resistance. For example, if the blood pressure signal and the PPG signal both exhibit high degrees of modulation, then it may be determined that there is a relatively low peripheral resistance. If, however, the blood pressure signal and the PPG signal both exhibit low degrees of modulation, then it may be determined that there is a relatively high peripheral resistance. Notably, the method determines cardiac output, and changes thereto, by accounting for changes in peripheral resistance, and is not tied solely to driving pressure. The method proceeds to 1110, in which a change in cardiac output over time is determined through the ratio.

Embodiments of the present disclosure provide a system and method of determining cardiac output and/or changes in cardiac output that may account for peripheral resistance, in contrast to previous model-based systems and methods. Embodiments of the present disclosure may be more accurate and reliable than previous systems that estimated cardiac output based solely on blood pressure, for example. Embodiments of the present disclosure provide a system and method of determining cardiac output through detecting blood pressure signal parameters through a blood pressure sub-system, and PPG signal parameters through a PPG sub-system, for example.

It will be understood that the present disclosure is applicable to any suitable physiological signals and that PPG and blood pressure signals are used for illustrative purposes. Those skilled in the art will recognize that the present disclosure has wide applicability to other signals including, but not limited to other physiological signals (for example, electrocardiogram, electroencephalogram, electrogastrogram, electromyogram, heart rate signals, pathological sounds, ultrasound, or any other suitable biosignal) and/or any other suitable signal, and/or any combination thereof.

Various embodiments described herein provide a tangible and non-transitory (for example, not an electric signal) machine-readable medium or media having instructions recorded thereon for a processor or computer to operate a system to perform one or more embodiments of methods described herein. The medium or media may be any type of CD-ROM, DVD, floppy disk, hard disk, optical disk, flash RAM drive, or other type of computer-readable medium or a combination thereof.

The various embodiments and/or components, for example, the control units, modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor may also include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

While various spatial and directional terms, such as top, bottom, lower, mid, lateral, horizontal, vertical, front, and the like may be used to describe embodiments, it is understood that such terms are merely used with respect to the orientations shown in the drawings. The orientations may be inverted, rotated, or otherwise changed, such that an upper portion is a lower portion, and vice versa, horizontal becomes vertical, and the like.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A system for determining cardiac output of a patient, the system comprising:
a first sub-system configured to detect a first physiological signal;
a second sub-system configured to detect a second physiological signal that differs from the first physiological signal, wherein the first and second sub-systems are separate and distinct from one another; and
a cardiac output determination module that is configured to determine the cardiac output based, at least in part, on the first and second physiological signals.

2. The system of claim 1, wherein the first sub-system comprises a blood pressure sub-system and the first physiological signal comprises a blood pressure signal, and wherein the second sub-system comprises a photoplethsymogram (PPG) sub-system and the second physiological signal comprises a PPG signal, and wherein the cardiac output determination module is configured to determine driving pressure from the blood pressure signal, and resistance to flow through an analysis of the PPG signal and the blood pressure signal.

3. The system of claim 2, wherein the blood pressure sub-system comprises a blood pressure monitor operatively connected to a blood pressure detection device, and wherein the blood pressure detection device is one or more of a non-invasive, minimally-invasive, or invasive blood pressure detection device.

4. The system of claim 2, wherein the PPG sub-system comprises a pulse oximetry sub-system.

5. The system of claim 1, wherein the cardiac output determination module is configured to determine the cardiac output based on an analysis of at least a first parameter of the first physiological signal and at least a second parameter of the second physiological signal.

6. The system of claim 5, wherein the at least a first parameter comprises one or more of a mean arterial pressure, a systolic pressure, or a diastolic pressure, and wherein the at least a second parameter comprises one or more of a change in amplitude, baseline, or frequency of the first and second physiological signals.

7. The system of claim 1, wherein the cardiac output determination module is configured to determine cardiac output based on a driving pressure determined from the first physiological signal, and a peripheral resistance determined from the first and second physiological signals.

8. The system of claim 1, wherein the cardiac output determination module is configured to determine a change in cardiac output over time based on a change in the first physiological signal over time and a relative change in the first and second physiological signals over time.

9. The system of claim 1, wherein the cardiac output determination module is configured to form a ratio of a change in the first physiological signal over time with respect to a change in the second physiological signal over time.

10. A method of determining cardiac output of a patient, the method comprising:
detecting a first physiological signal of the patient with a first sub-system;
detecting a second physiological signal of the patient with a second sub-system that is separate and distinct from the first sub-system, wherein the second physiological signal differs from the first physiological signal; and
determining the cardiac output based, at least in part, on the first and second physiological signals, with a cardiac output determination module.

11. The method of claim 10, wherein the first physiological signal includes a blood pressure signal, and the second physiological signal comprises a photoplethysmogram (PPG) signal, and wherein the method comprises determining driving pressure from the blood pressure signal, and determining resistance to flow through an analysis of the PPG signal and the blood pressure signal.

12. The method of claim 10, wherein the determining operation comprises analyzing at least a first parameter of the first physiological signal and at least a second parameter of the second physiological signal.

13. The method of claim 12, wherein the at least a first parameter comprises one or more of a mean arterial pressure, a systolic pressure, or a diastolic pressure, and wherein the at least a second parameter comprises one or more of a change in amplitude, baseline, or frequency of the first and second physiological signals.

14. The method of claim 10, wherein the determining operation comprises determining a driving pressure from the first physiological signal, and determining a peripheral resistance from the first and second physiological signals.

15. The method of claim 10, wherein the determining operation comprises determining a change in cardiac output over time based on a change in the first physiological signal over time and a relative change in the first and second physiological signals over time.

16. A tangible and non-transitory computer readable medium that includes one or more sets of instructions configured to direct a computer to:
detect a first physiological signal of a patient with a first sub-system;
detect a second physiological signal of the patient with a second sub-system that is separate and distinct from the first sub-system, wherein the second physiological signal differs from the first physiological signal; and
determine the cardiac output based, at least in part, on the first and second physiological signals, with a cardiac output determination module.

17. The tangible and non-transitory computer readable medium of claim 16, wherein the first physiological signal includes a blood pressure signal, and the second physiological signal comprises a photoplethysmogram (PPG) signal, and the tangible and non-transitory computer readable medium is further configured to determine driving pressure from the blood pressure signal, and determine resistance to flow through an analysis of the PPG signal and the blood pressure signal.

18. The tangible and non-transitory computer readable medium of claim 16, further configured to direct the computer to analyze at least a first parameter of the first physiological signal and at least a second parameter of the second physiological signal to determine the cardiac output.

19. The tangible and non-transitory computer readable medium of claim 16, further configured to direct the computer to determine a driving pressure from the first physiological signal, and determine a peripheral resistance from the first and second physiological signals.

20. The tangible and non-transitory computer readable medium of claim 16, further configured to direct the computer to form a ratio of a change in the first physiological signal over time with respect to a relative change in the first and second physiological signals over time.
